(19) United States
(12) Patent Application Publication
(10) Pub. No.: US 2005/0085840 A1
(43) Pub. Date: Apr. 21, 2005

(54) Lancet assembly

(75) Inventors: Patrick Yi, Singapore (SG); Kook-Ting Foo, Singapore (SG)

Correspondence Address: TROUTMAN SANDERS LLP, BANK OF AMERICA PLAZA, SUITE 5200, 600 PEACHTREE STREET , NE, ATLANTA, GA, 30308-2216, US

(73) Assignee: SURGILANCE PTE LTD

(21) Appl. No.: 10/965,555

(22) Filed: Oct. 14, 2004

Related U.S. Application Data
(60) Provisional application No. 60/511,230, filed on Oct. 15, 2003.

(51) Int. Cl. A61B 17/32

(52) U.S. Cl. 606/182

(57) ABSTRACT

A lancet assembly having a lancet holder and a triggler enclosing a lancet structure. The triggler is partially inserted into a lancet holder from the distal end. The holder is provided with a rigid internal spring holder to receive the spring-loaded lancet structure. The lancet structure is provided with a body coupled to a spring which extend from the proximal end of the body. The spring has a linear axis of compression which coincide with the longitudinal axis of the lancet assembly. A lancet is attached to the body with the sharp tip pointing towards the distal end. The triggler interacts with the lancet holder via a triggering element to maintain the spring in a compressed state such that the lancet structure is in a stable standby position which is not easily triggered by accidental bumps on the assembly. Upon firing, a second spring dampens the extension of the sharp tip, and ultimately assists in the retraction of the tip back into the assembly.

### RELATED US APPLICATION DATA

This application claims priority from U.S. Provisional Application No. 60/511,230 filed Oct. 15, 2003.

### FIELD OF THE INVENTION

The present invention is related to finger-pricking devices. In particular, the present invention is related to lancets for medical use.

### BACKGROUND OF THE INVENTION

Lancets or finger-pricking devices are used in the medical field for applications such as skin incisions and blood drawing. In some lancet assemblies, the blade or needle is kept in a standby position until it is triggered by the user, who is typically medical personnel in charge of drawing the blood from a patient. In other lancet assemblies, the user has to manually set the assembly to an armed position before firing can be triggered. Upon triggering, the blade fires onto the skin of the patient, for example on the finger, and makes an incision.

Such prior art lancet assemblies must be sterilized before use, and the lancet maintained under sterile conditions until use. Beneficially, lancet assemblies should be disposable (single use) to eliminate the chances of disease transmission due to the blade being used on more than one person. In this regard, the lancet should ideally be designed for only one firing, and have safety features to prevent reuse. Other features which are desirable include safety features to reduce the chance of the lancet accidentally misfiring and pricking someone.

It is an object of the present invention to provide a safe and reliable lancet assembly.

### SUMMARY OF THE INVENTION

Briefly described, in a preferred form, the present invention is an improvement over the conventional lancet assembly by providing in one aspect a lancet assembly having a distal end, a proximal end and a longitudinal axis running there between. The triggler is partially inserted into a housing from the distal end. The housing is provided with a rigid internal spring holder to receive a spring-loaded lancet structure. The lancet structure is provided with a body coupled to a spring which extend from the proximal end of the body. A lancet is attached to the body with the sharp tip pointing towards the distal end. The triggler interacts with the housing via a triggering element to maintain the spring in a compressed state such that the lancet structure is in a stable standby position which is not easily triggered by accidental bumps on the assembly. The standby position is automatically maintained, and an additional arming step is eliminated. In another aspect, the assembly is provided with a safety feature to prevent tampering of the lancet assembly. In yet another aspect, the spring is secured to the end wall of housing such that dislocation caused by the sudden expansion force of the firing process is prevented. Furthermore, the spring is chosen to over-extend upon firing, such that the sharp tip of the lancet can jab a patient's skin held outside the housing upon firing, but the sharp tip is withdrawn safely back into the lancet assembly when the spring is recoiled into the resting position.

In another aspect, a method is provided for finger pricking using a lancet assembly with a distal end and a proximal end. This lancet assembly contains a lancet with a sharp tip that fires outwards from the distal end when the assembly is compressed inwards from the same distal end. The method involves placing the distal end of the lancet assembly onto a finger to be pricked, and pressing the assembly into the finger from the proximal end. This compresses the assembly and causes firing of the lancet whereby the finger is pricked.

In the preferred embodiment, the housing is provided with an open distal end and a closed proximal end defined by an end wall. Side walls extend from the end walls to form a box-like exterior casing. A mating element, preferably disposed on the interior surface of the side walls, is provided to interact with the triggler. The housing also contains an elongated spring holder having an open first end and a second end attached to and extending from the end wall of the housing. The supporting structures between the first end and the second end define an interior space wherein the lancet structure is received. A receiving element is provided on the spring holder for mating with a guiding element on the lancet structure such that the lancet can move along the longitudinal axis. A securing element is provided near the proximal end of the housing for securing the proximal end of the spring from dislocation during firing.

The triggler contains an end wall for closing the open distal end of the housing. This triggler end wall contains an aperture where through the sharp tip of the lancet fires when triggered. A stabilizing element, extending from the triggler end wall, is inserted between the side walls of the housing and the supporting structures of the spring holder, allowing sliding movement of the triggler relative to the housing along the longitudinal axis. This sliding movement is limited by a safety catch on the triggler which is designed to engage the mating element of the housing such that the triggler is maintained between a standby position and a firing position. A triggering element is also provided in the triggler for engagement with the guiding element of the lancet structure. When the triggler and the triggering element are in the standby position, the guiding element is forced towards the proximal end of the housing, such that the spring is maintained in the standby position of a compressed high potential energy state. When a compression force is applied to the triggler end wall in the direction of the proximal end, the triggering element may be moved into a firing position in which it is disengaged from the guiding element. Once the guiding element is no longer held in the standby position, the potential energy stored in the compressed spring is released and the spring extends, forcing the sharp tip of the lancet structure to fire through the aperture of the triggler end wall.

These and other objects, features and advantages of the present invention will become more apparent upon reading the following specification in conjunction with the accompanying drawings.

### BRIEF DESCRIPTION OF THE DRAWINGS

FIG. 1 is a perspective view of the housing with the side walls cut away to expose the internal features.

FIG. 2 is the bottom view of the triggler according to the present invention.

FIG. 3A is an exploded perspective view of the lancet assembly according to the present invention.

FIG. 3B is an exploded cross-sectional view of the lancet assembly along line A-A according to the present invention.

FIGS. 4A and B are longitudinal cross-sectional views along line A-A of the lancet assembly in the after use and firing positions respectively according to the present invention.

FIGS. 4C and D are longitudinal cross-sectional views along line A-A of the lancet assembly in the standby position with (FIG. 4C) or without (FIG. 4D) the protective cap according to the present invention.

FIG. 5 is an illustration of a two-spring embodiment of the present invention.

FIGS. 6A-F are longitudinal cross-sectional views of the use of the lancet assembly incorporating the springs of FIG. 5.

FIG. 7 is an illustration of a second two-spring embodiment of the present invention.

### DETAILED DESCRIPTION OF A PREFERRED EMBODIMENT

Referring now in detail to the figures, wherein like reference numerals represent like parts throughout the several views, the present lancet assembly comprises a housing 2, firing element 4, lancet system 6, triggering element 8, and release system 10. The device is preferably pre-armed; that is, the device needs no arming by the user, just a light push on the triggering element 8 (preferably triggler 50) activates the release system 10, which releases the firing element 4 (preferably a spring 45), extending the lancet system 6, and the skin is pierced.

The present lancet assembly has a longitudinal axis defined by the axis of compression of the spring 45. The triggler 50 (or triggering device) and housing 2 interact by keeping the spring 45 in a standby, compressed, pre-armed state. Upon compression of the assembly along the longitudinal axis by the user pressing the end wall of the triggler onto the skin of a patient, the lancet will be fired to pierce the skin.

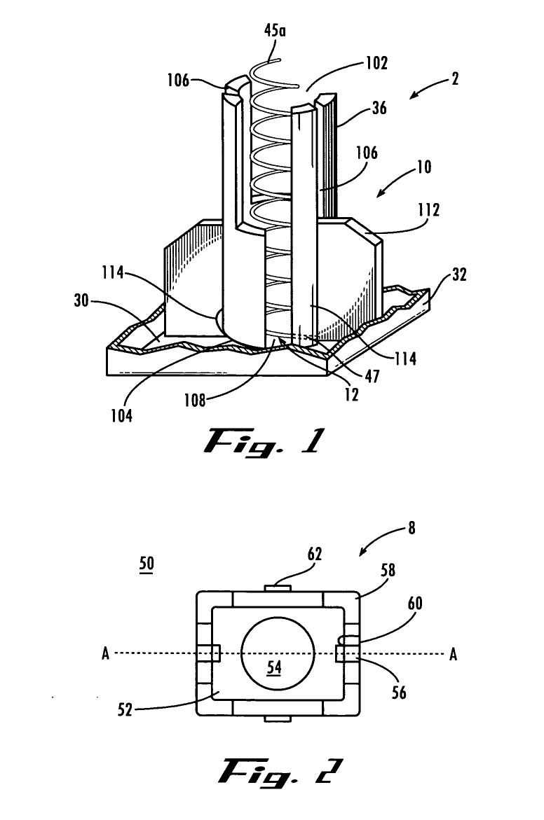

FIG. 1 illustrates an internal view of housing 2, and shows an embodiment of the housing 2 with the side walls cut away to reveal the spring holder 36. A spring 45a is shown in dotted line. In this embodiment, the spring holder 36 is generally shaped into a cylinder with an open first end 102, and a second end 104 attached to the end wall 30 of the housing 2. Two slits 106, running from the open first end towards the proximal end are provided on the side wall of the spring holder. In this embodiment, a wider slot 108 is further cut into the side wall of the spring holder 36. To strengthen the side walls of the spring holder, two ridges 112 are provided to connect the side walls of the spring holder and the housing 2. This improves the rigidity of the spring holder, and hence the reliability and accuracy of the firing procedure. An additional pair of lugs 114 is provided at the proximal end of the spring holder juxtaposing the two sides of slot 108. These lugs can interact with a unshaped extension 47 in the spring 45, although the spring 45 need not incorporate such an extension 47.

The housing 2 is preferably made from a rigid plastic material with good mechanical strength such as polycarbonate material in order to withstand the high energy firing process of the metal spring.

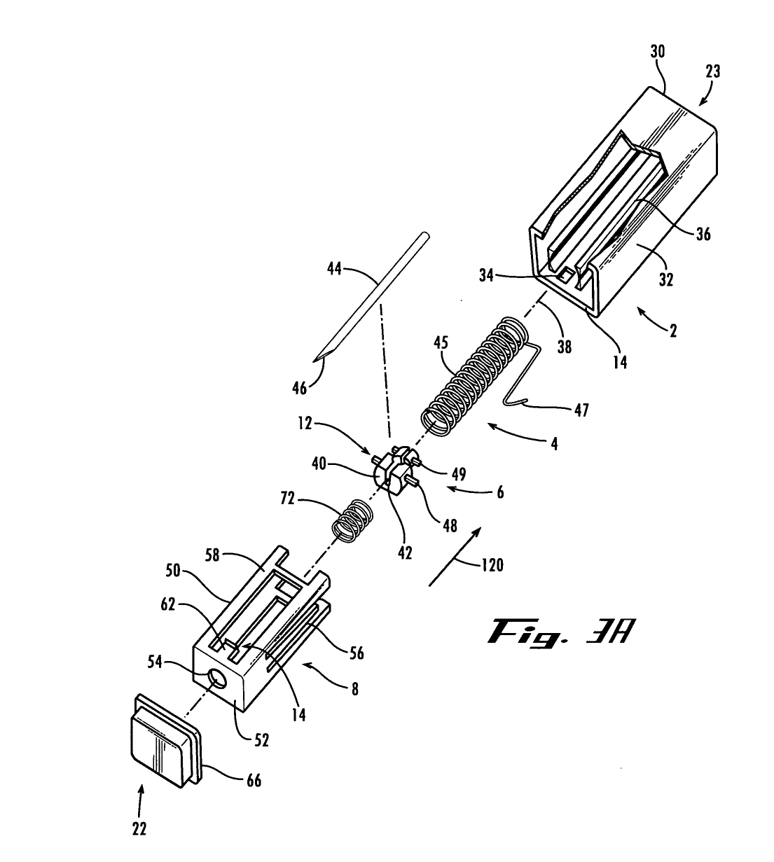

FIG. 2 shows detailed features of the triggering element 8, comprising triggler 50. In this embodiment, the triggler is of a rectangular shape having an end wall 52 with aperture 54. Side walls 58 extending from the four corners of end wall 52 to form a stabilizing element. The triggering element, in the form of actuating ribs 56 extends from two opposing sides of end wall 52. A small protrusion, in the form of wedge 60, is provided at the end of each actuating rib 56. Wedge 60 protrudes into the interior of the triggler. Two L-shaped clips 62 further extend from opposing sides of end wall 52. The triggler is preferably made from a slightly flexible plastic, such as Acrylonitrile Butadiene Styrene (ABS) material to allow the actuating ribs to bend with the right resistance.

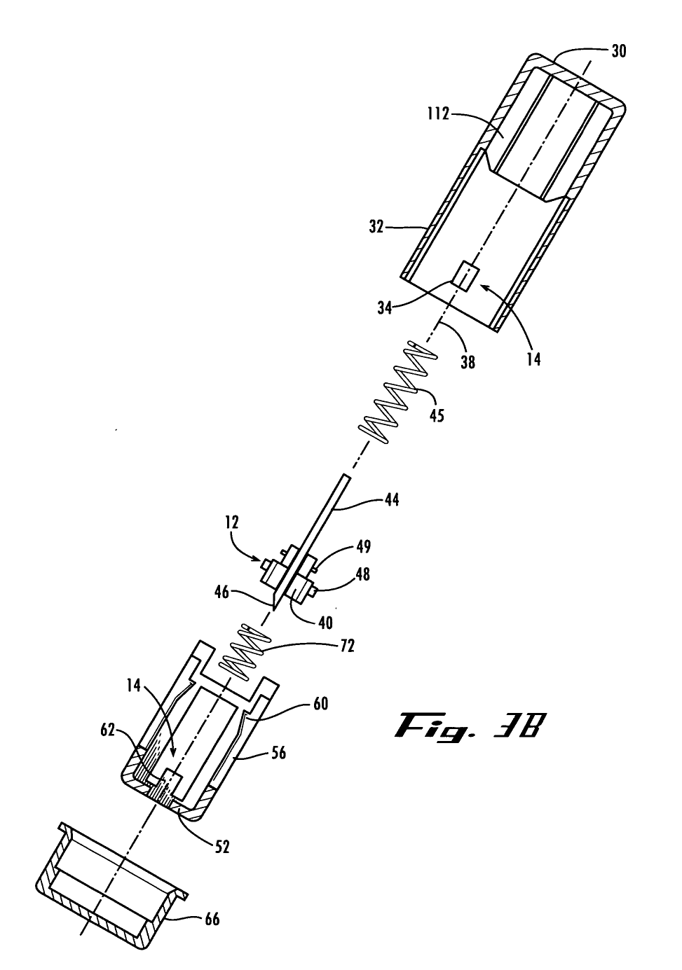

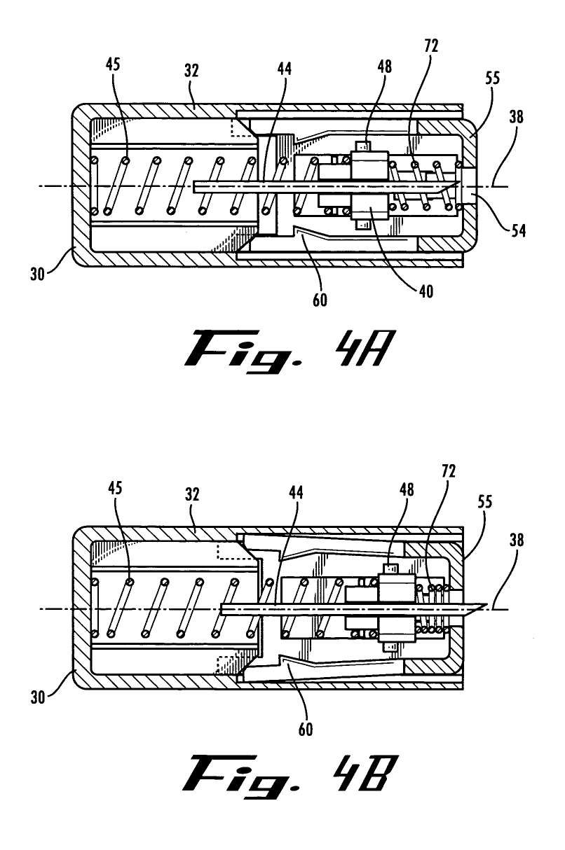

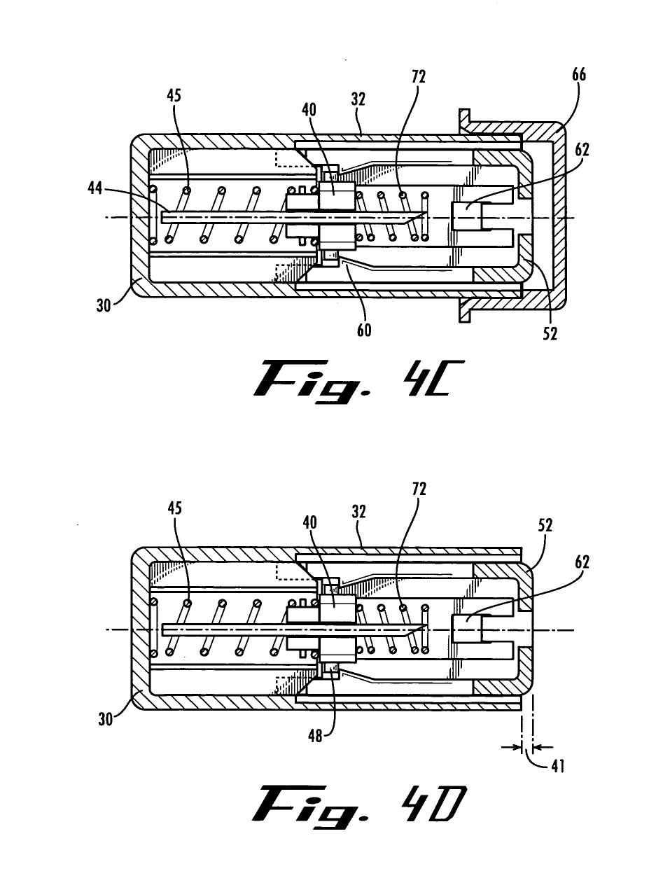

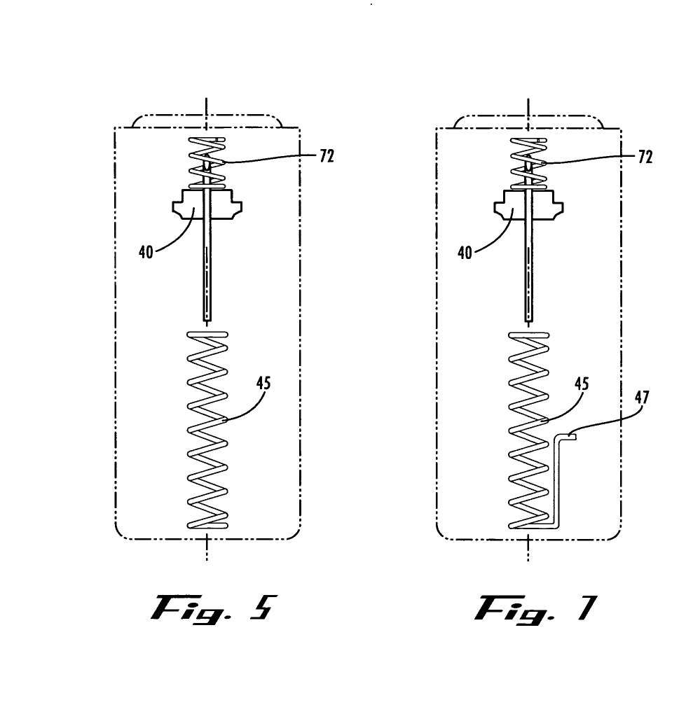

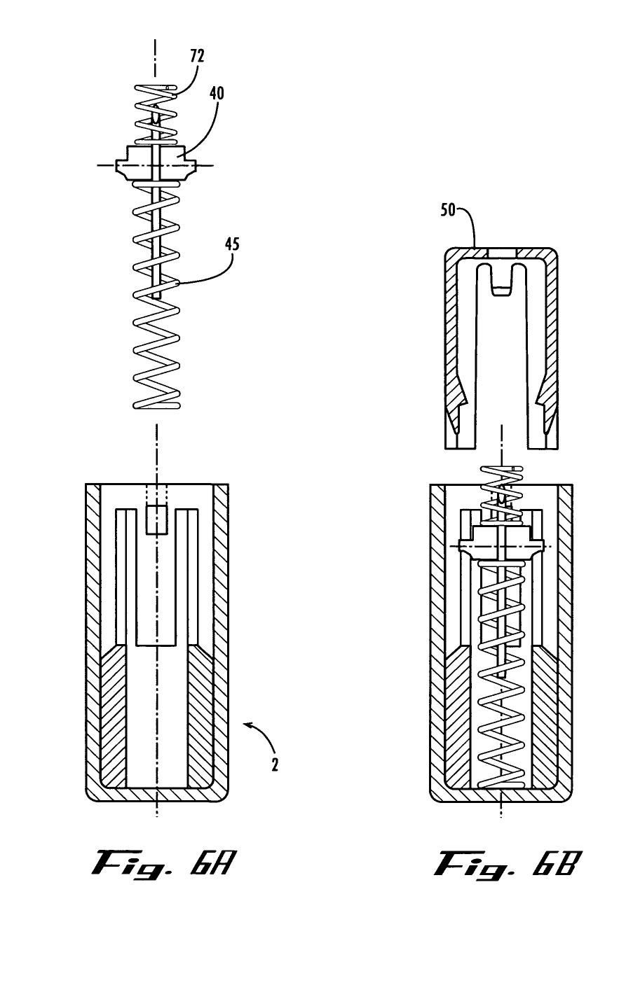

FIGS. 3A, 3B and 4A-D illustrate how the present device, having a longitudinal axis 38, a distal end 22 and a proximal end 23, is assembled to provide the desired features. The lancet system 6 includes a lancet body 40, and a lancet 44 with a sharp tip 46. In this embodiment, the firing element 4 for the lancet system 6 is a spring 45 being a helical metal coil that has a U-shaped extension 47 at the proximal end, although the spring 45 need not incorporate such an extension 47, as shown in FIG. 5. Lancet body 40 is provided with channel 42 for receiving and securing the lancet such that the sharp tip 46 of the lancet is at the distal end. Guiding pins 48 can provided on opposing sides of body 40. Securing pins 49 can be provided, and be extended from the proximal end of body 40 for securing spring 45 onto the body. In another embodiment of the present device, the spring 45 is not anchored or otherwise fixed to the lancet system 6. In yet another embodiment, the spring 45 is completely loose within the housing 2, anchored neither to the housing 2 nor to the lancet body 40.

The present invention may further include a guide system 12 comprising the interplay of pins of the lancet system 6, and the housing 2, to provide true and straight firing of the assembly, should such firing be warranted. In some embodiments, rotational movement of the lancet, for example, might be beneficial.

The assembled lancet structure is received within spring holder 36 of housing 2 with the firing element 4, or spring 45, pressed onto end wall 30 of housing 2. The guide system 12 comprising guiding pins 48 are slotted into slits 106 such that lateral movement and rotational movement around the longitudinal axis are prevented. The spring 45 is fitted onto the lancet body 40 such that the U-shaped extension 47 extends through slot 108 and wraps around either one of lug 114. This arrangement prevents the spring from being easily dislocated from the spring holder during the firing process.

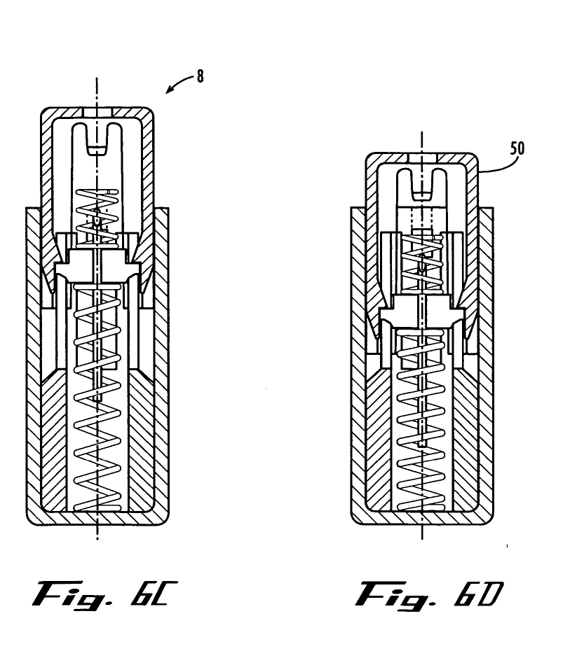

In an alternative embodiment, the spring 45 is not anchored to the housing 2, either fixedly or by wrapping an element of the spring 45 around a lug 114 of the housing 2. Such a loose end of the spring is shown in FIGS. 5 and 6A-F, where 45 is not anchored to housing 2.

The side walls 58 of triggler 50 are slidably inserted into the space between the spring holder 36 and the side walls 32 of the housing 2 such that the small wedges 60 of actuating ribs 56 slide through slits 106 and press the guiding pins towards the proximal end of the housing 2.

The present invention can further comprise retention system 14, to limit the reuse of the device after firing. The retention system 14 can comprise an L-shaped clip 62 on the triggler 50 that is adapted to engage the elongated recess 34 in the housing 2 to limit the longitudinal movement of the triggler relative to the housing 2. In this way, the triggler is caught to the housing 2 directly, and the retention system 14 need not incorporate any other element of the present invention to limit reuse, while prior art devices require the interplay of the spring, for example, to perfect non-reuse.

During assembly, the triggler is pressed down toward the housing 2 in the direction shown by arrow 120, and in the process, the guiding pins, are forced down concomitantly by the small wedges on the actuating ribs. This compression force compresses the spring 45.

To fire the device, the user lightly pushes the triggler into the housing 2, the resistance of the spring 45 to further compression becoming greater. When the resistance of the spring 45 exceeds the mechanical strength of the actuating ribs to push on the guiding pins, the actuating ribs are bent outwards relative to the longitudinal axis of the housing 2, releasing the compression of the lancet structure and resulting in firing of the lancet.

In another embodiment of the present invention, the design and material of the actuating ribs 56 and their play with the housing 2 enables the spring 45 to have only one energization level, and the pushing of the triggler imparts no further compression of the spring when the triggler is activated by the user. That is, the pre-armed device has simply one energization of the spring 45, the compression of the spring upon delivery to a user. When the user pushes on the triggler, the spring is not compressed to a second energization level, but remains at the same compressive state until the ribs are bent outward, releasing the spring 45.

In the preferred embodiment, a cap 66 is provided to interact with the side walls 32 of the housing 2 to maintain sterility and to protect users in cases of accidental firing. The cap is preferably an air-seal or sterile cap.

In the standby position, the resistance of the spring 45 to compression forces clip 62 towards the distal end of recess 34. In a preferred embodiment, the length of actuating ribs 56 is designed to allow the end wall of the triggler to extend a short distance 41 from the side walls 32 of the housing 2 when the assembly is in the standby position, for example, 12 mm from the distal end. This short distance means that the triggler is well protected from firing due to accidental bumping.

Furthermore, due to the lack of lateral movement, the direction of the external pressure has to be very close to the longitudinal axis of the assembly in order for the triggler to be compressed sufficiently for firing to occur. The mechanical strength of the actuating rib may be designed such that deformation (i.e. firing) only occurs when sufficient pressure is provided on the triggler end wall. In addition, the spring may be chosen such that triggering only occurs when the triggler is flush against the edge of the proximal end of the housing 2, or even pushed slightly inside.

All these parameters may be varied to reduce the chance of accidental firing without undue experimentation. When the user wants to prick the finger of the patient, he/she presses the end wall 52 of the triggler against the skin of the finger. This compression force pushes clip 62 towards the proximal end of the recess and the triggler towards the proximal end of the housing 2, increasing the compression of the spring in one embodiment, or, in another preferred embodiment, not further compressing the spring, but only deforming the ribs so no vertical vector of force is imparted to the spring, the ribs deforming laterally.

In the embodiment of the device that imparts a further compression upon the spring 45, the triggler insertion causes the spring to generate an opposing extension force, which pushes against wedges 60 of the actuating ribs 56. Ribs 56 are designed to bend outwards into the firing position with this additional pressure, in which wedges 60 disengages from guiding pin 48, causing the spring to fire into a fully extended position and the lancet to jab the skin of the patient. In the embodiment of the device that imparts no further compression upon the spring, the triggler insertion causes only the deformation of the ribs 56, wherein at some point, the wedges 60 disengages from guiding pin 48, causing the spring to fire into a fully extended position and the lancet to jab the skin of the patient.

It is clear that from the above description, the triggering mechanism is from the interaction with the patient's skin. As a result, if the patient pulls back his hand just when the user is about to press down the triggler, triggering would not occur.

In one embodiment of the device, the slot 108 and lug 114 at the proximal end of spring holder acts as the securing element of the spring such that after firing, the distal end of the spring recoils towards the proximal end. In this way, the sharp tip of the lancet is retracted completely back into the housing 2 after firing. This is a highly desirable safety feature. In another embodiment, even though the spring is not anchored to the housing, the interplay of the various elements of the device nonetheless enable the distal end of the spring to recoil towards the proximal end, providing the same level of safety.

FIGS. 5, 6A-F and 7 illustrate yet another embodiment of the present invention, wherein the retention system 14 and/or release system 10 further comprises spring 72. Spring 72 can be thought of as assisting in the retention system 14 by retaining the lancet system in the housing after firing, and can also be thought of assisting the release system 10 by making the projection of the lancet system from the housing momentary, by forcing the lancet back into the body after firing.

Upon firing, the second spring 72 dampens the force of extension of the sharp tip of the lance, and ultimately assists in the retraction of the tip back into the assembly.

Just as spring 45 can be anchored or otherwise fixed to either or both the housing 2 or lancet body 40, so to can spring 72 be anchored, or not, to either or both the lancet body 40, or triggler end wall 52.

In a preferred embodiment of the device of FIGS. 5, 6A-F and 7, the spring 45 is completely loose within the housing 2. The spring 45 is compressed and pre-energized upon manufacture, and the interplay of the forces imparted by springs 45 and 72 maintain the device in the armed condition. When the device is fired, spring 45 provides only a shooting force to the lancet, as the spring 45 is not attached to the lancet, and thus can play no or only a minimal role in retracting the lancet. That is, the spring 45 cannot, itself, make the lancet firing momentary.

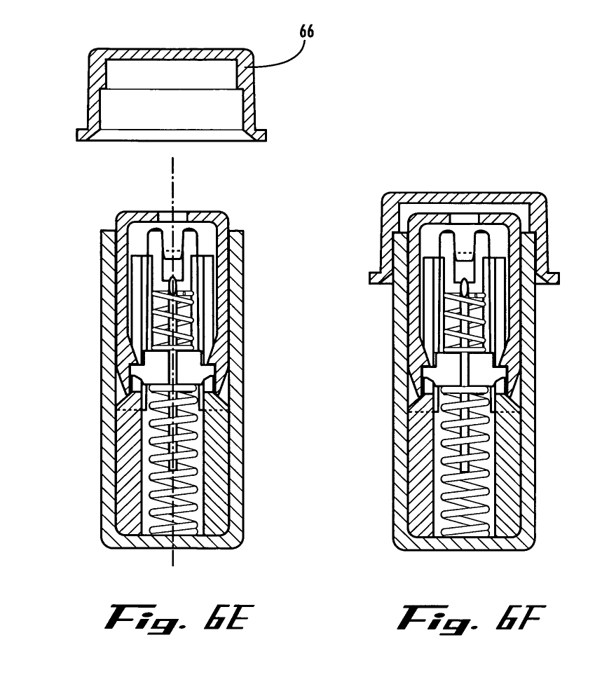

Spring 72 is responsible for the majority, if not all, of the retraction force, to bring the lancet back into the device after firing, and making the projection of the lance tip momentary. FIGS. 6A-F illustrate, in a similar fashion as FIGS. 4A-D, the various steps of the two-spring embodiment of the present invention. Yet, FIGS. 6A-F show the device when a push on the triggler imparts further compressive force upon spring 45. It is noted that in another embodiment of the invention, the triggler imparts no further energization of spring 45, and only fires the device by having all the vertical force of the triggler push translated into deforming the ribs of the triggler. FIG. 7 illustrates the two spring embodiment, with spring 45 having the extension 47, not present in FIG. 5.

The stabilizing element in the present invention, described as side walls 68 at the four comers of the triggler 50 in above embodiment, serves not only as a structure to allow interaction between the triggler and the housing 2, but also a structure to prevent lateral movements there between. As a result, only firm compressional pressure almost directly along the longitudinal axis is required for firing to occur, and accidentally knocking the triggler in any other direction is not likely to cause firing. Besides having side walls on all four comers, other embodiments may be possible, such as rigid structures symmetrically extending from the end walls of the triggler. The tight association between the triggler and the housing 2 also means that the lancet structure is completely protected and enclosed, and it is extremely difficult to dismantle the assembly without breaking some parts.

The securing element may be other structures in the molded plastic spring holder that allows interaction of the proximal end of the spring with the proximal end of the housing 2. This securing element allows a metallic helical spring with superior springback properties to be used instead of plastic springs that may be fabricated as an integral part of the spring holder.

Besides a recess on the interior side wall of the housing 2, other structures, such as an elongated aperture or a bracket in the side wall may also serve as the mating element. A structure which is only accessible from the interior is, however, preferred as it prevents external tampering.

While the invention has been disclosed in its preferred forms, it will be apparent to those skilled in the art that many modifications, additions, and deletions can be made therein without departing from the spirit and scope of the invention and its equivalents, as set forth in the following claims.

1. A lancet assembly comprising:
a housing;
a firing element containable within the housing;
a lancet system capable of being extended outside the housing by the firing element;
a triggering element to trigger the firing element;
and a release system to enable the extension of the lancet system beyond the housing.

2. The lancet assembly of claim 1, further comprising a guide system to guide the extension of the lancet system beyond the housing.

3. The lancet assembly of claim 1, further comprising a retention system disable the device from reuse once the lancet system has extended beyond the housing.

4. The lancet assembly of claim 1, the firing element being anchored to the housing.

5. The lancet assembly of claim 1, the firing element being unanchored to the housing.

6. The lancet assembly of claim 1, the firing element being anchored to the lancet system.

7. The lancet assembly of claim 1, the firing element being unanchored to the lancet system.

8. The lancet assembly of claim 1, the firing element being pre-energized, and moved to a second energizaton level upon activation of the triggering element.

9. The lancet assembly of claim 1, the firing element being pre-energized, and remaining at the same pre-energized level upon activation of the triggering element.

10. A lancet assembly comprising:
a housing;
a firing element containable within the housing;
a lancet system capable of being extended outside the housing by the firing element;
a triggering element to trigger the firing element;
a release system to enable the momentary extension of the lancet system beyond the housing; and
a retention system to disable the device from reuse once the lancet system has extended beyond the housing.

11. The lancet assembly of claim 10, the firing element being a first spring, and the retention system including a second spring.

12. The lancet assembly of claim 11, the firing element being pre-energized, and remaining at the same pre-energized level upon activation of the triggering element.

13. The lancet assembly of claim 12, the retention system further comprising at least one clip extending from the triggering element, the at least one L-shaped clip engaging a recesses of the housing.

14. A lancet assembly comprising:
a lancet system including a lancet body having a proximal end, a distal end and guiding elements, the lancet system further including a lancet with a sharp tip that extends from the distal end of the lancet body;
a housing including a lancet holder and a spring holder;
the lancet holder including an open distal end, a lancet holder end wall at a proximal end, and lancet holder side walls extending from the lancet holder end wall, the lancet holder side walls having a mating element including recesses in the interior of the lancet holder side walls; and
the spring holder including an open first end, a second end attached to the lancet holder end wall, and supporting structures there between defining an interior space, the lancet system received within the interior space with the sharp tip proximate the open first end, the supporting structures further provided with receiving elements for engagement with the guiding elements of the lancet system;
a firing element containable within the housing, the firing element being a first spring located loosely between the proximal end of the lancet body and the second end of the spring holder, the first spring having a proximal end and a linear axis of compression, the supporting structures of the spring holder having the receiving elements for engagement with the guiding elements of the lancet system to allow movement of the lancet body along the axis of compression;
a triggering element to trigger the firing element, the triggering element including;
a triggler end wall for closing the distal end of the lancet holder and having an aperture wherethrough the sharp distal tip of the lancet fires when triggered;
a stabilizing element, extending from the triggler end wall and inserted between the side walls of the lancet holder and the supporting structures of the spring holder for sliding movement along the axis of compression, the stabilizing element further limiting movement of the triggler lateral to the axis of compression; and
a safety catch for maintaining the triggler between standby and firing positions, wherein the safety catch includes L-shaped clips extending from two opposing sides of the triggler end wall, the L-shaped clips engaging the recesses of the mating element of the lancet holder;
and a release system to enable the extension of the lancet system beyond the housing, the release system including triggering elements and a second spring, the triggering elements for engagement with the guiding elements of the lancet system, the triggering elements in the standby position maintaining the first spring in a compressed state by forcing the guiding elements toward the proximal end of the lancet holder, the triggering elements further movable to the firing position when a compression force is applied to the triggler end wall, the triggering elements in the firing position being disengaged from the guiding elements such that the first spring is released from the compressed state and the sharp distal end of the lancet system is fired through the aperture of the triggler end wall, the release system providing the momentary extension of the sharp tip of the lancet beyond the aperture of the triggler end wall with the provision of the second spring located loosely between the distal end of the lancet system, and the triggler end wall.

15. The lancet assembly of claim 14, wherein the guiding elements comprises a plurality of guiding pins extending laterally from at least two sides of the lancet body.

16. The lancet assembly of claim 15, wherein the receiving elements of the supporting structures of the spring holder comprise a plurality of longitudinal slits within the supporting structures wherethrough each of the guiding pins extend.

17. The lancet assembly of claim 14, wherein the triggering elements include a pair of actuating ribs with wedged ends extending from two opposing sides of the triggler end wall; and
wherein the guiding elements include a pair of guiding pins extending laterally from two opposing sides of the lancet body, the triggler inserted into the lancet holder such that the wedged ends engage the guiding pins, the actuating rib in the armed position maintaining the first spring in a high potential energy compressed state by forcing the guiding pins towards the proximal end of the lancet holder, the actuating rib further bendable to the firing position when the compression force is applied to the triggler end wall.

18. The lancet assembly of claim 14, wherein the lancet holder further comprises ridges attached to the spring holder, the lancet holder to strengthen the supporting structures.
